(19) United States
(12) Patent Application Publication
(10) Pub. No.: US 2004/0142885 A1
(43) Pub. Date: Jul. 22, 2004

(54) Biological organism for preparing pharmaceutical compositions for treating mammals

(76) Inventors: Stephane Paul, Strasbourg (FR); Etienne Regulier, Evian-Les-Bains (FR)

Correspondence Address: D Douglas Price, Steptoe & Johnson, 1330 Connecticut Ave N W, Washington, DC, 20036, US

(21) Appl. No.: 10/470,244

(22) PCT Filed: Jan. 28, 2002
(86) PCT No.: PCT/FR02/00330

(30) Foreign Application Priority Data
Jan. 26, 2001 (EP) 01440017.0

(51) Int. Cl. A61K 48/00; C07H 21/04

(52) U.S. Cl. 514/44; 536/23.53

(57) ABSTRACT

The invention concerns a biological organism comprising at least: (i) a nucleic acid coding for all or part of an antibody, said antibody being expressed at the surface of a host cell and capable of being fixed to a polypeptide present at the surface of a cytotoxic effector cell or of a T-helper lymphocyte and involved in the process of activating such a cell and (ii) a nucleic acid coding for all or part of a polypeptide involved in the stimulation of an immune response or in attraction to the site of expression and activation of cytotoxic effector cells or of T-helper lymphocytes, said polypeptide being selected among chemokines and co-stimulation molecules. The invention also concerns a host cell and a pharmaceutical composition containing said biological organism and their therapeutic or prophylactic uses.

The present invention relates to the field of gene therapy applied to specific or nonspecific immunotherapy (or gene immunotherapy), more especially within the context of treating diseases the responsible agent for is a pathogenic organism such as, in particular, a bacterial, parasitic or viral agent, or within the context of treating cancer or prion diseases. More especially, the invention relates to a biological material which comprises at least (i) one nucleic acid encoding all or part of an antibody and (ii) one nucleic acid encoding all or part of a polypeptide which is involved in stimulating the immune response and/or influencing the cytotoxic effector cells and/or the T helper lymphocytes. The present invention also relates to a host cell and to a composition which comprises said biological material, and also to their use for medical purposes, more especially therapeutic or prophylactic purposes.

For a long time now, gene therapy has been proposed as a means for correcting disorders which are observed within the context of the genetic diseases. These diseases are explained, in particular, by there being a malfunction in the expression of specific genes or by nonfunctional mutated polypeptides being expressed in at least one cell type. Gene therapy consists in transferring, to the patient to be treated, the genetic information which is capable of correcting the observed defect. This genetic information can be, for example, the gene encoding the CTFR protein, in the case of cystic fibrosis, or the gene encoding dystrophin in the case of Duchenne's dystrophy. In this approach, the genetic information is either introduced in vitro into a specific host cell removed from the organ, with the modified cell then being reintroduced into the body (ex vivo method) or else introduced directly in vivo into, or in the vicinity of, the appropriate tissue (e.g. affected organ). A large number of publications describe the implementation of gene therapy protocols whose purpose is to obtain expression of a relevant protein in the host cells by means of introducing the corresponding genetic information.

However, the benefit of this type of approach is not confined to treating disorders which are purely of genetic origin; the approach can also make it possible, or contribute to making it possible, to reduce or eliminate tumors, isolated tumor cells, pathogenic agents, such as bacterial, parasitic or viral agents, or cells which are infected with such pathogenic agents. Failing that, such a treatment makes it possible to delay the progress of the stated disorders or disorders caused by the abovementioned agents.

In the particular case of cancers, Hellstrom et al. (1969, Adv. Cancer Res. 12, 167-223) have shown that the defence of the body to tumors is specifically based on the immune response which brings into play the T lymphocytes, in particular the cytotoxic T lymphocytes. However, a large number of studies have shown that the majority of the specific or nonspecific immune effectors are ineffective in eliminating, or even arresting the progress of, a tumor. It is consequently desirable to have available a method for stimulating the immune response which is directed against tumors or their antigens, more specifically the response which brings about the involvement of the cytotoxic lymphocytes, i.e. CTLs, in order to have available more effective methods for preventing or treating cancerous states. In an identical manner, it has been shown that the immune system is frequently ineffective in the case of infections, in particular viral infections, as is illustrated by infections due to HIV (human immunodeficiency virus).

The immune response which is directed against a specific antigen can be divided into two distinct categories: one bringing antibodies into play (humoral immune response) and the other bringing into play cytotoxic effector cells, such as macrophages, cytotoxic lymphocytes (CTLs) or killer cells (NKs and NKTs), and also T helper lymphocytes, in particular LTCD4 cells (cellular immune response). More specifically, the two types of response differ in that the antibodies recognize the antigens in their three-dimensional form whereas T lymphocytes, for example, recognize peptide portions of said antigens, with these portions being combined with glycoproteins which are encoded by the genes of the major histocompatibility complex (MHC), in particular in genes of the type I major histocompatibility complex, which genes are expressed ubiquitously on the surface of the cells, or genes of the type II major histocompatibility complex, which genes are expressed specifically on the surface of cells which are involved in antigen presentation (APCs). These fundamental elements of the immune response are well known to the skilled person.

The cellular immune response is characterized in that, following a well-known activation phenomenon (for a review, see Alberola-Ila, 1997, Annu. Rev. Immunol. 15, 125-154), the CD4+ T cells (helper T cells) produce cytokines which, in turn, induce the proliferation of APC cells which are able to produce said cytokines; the cellular differentiation of B lymphocytes which are able to produce specific antibodies; and the stimulation of cytotoxic T lymphocytes (CTLs). According to a second aspect of the cellular immune response, the cytotoxic effector cells, such as CD8+ lymphocytes (CTLS), are activated a) after interaction with antigenic peptides which are mounted on, and presented by, the glycoproteins which are carried ubiquitously by cells and encoded by genes belonging to the MHC I system, and b) possibly by the cytokines produced by the CD4+ cells. The CTLs which have thus been activated are then able to destroy the cells which are expressing said antigenic peptide.

It has been proposed that the methods of gene therapy, which are already well-known, should be adapted and that genes encoding immunostimulators which are capable of inducing or activating a cell-mediated immune response against a tumor should be transferred into host cells, more specifically cancer cells (immunotherapy). A large number of antineoplastic approaches have been described to date, with these approaches being based on using genes encoding cytokines (Colombo et al., 1994, Immunology Today, 15, 48-51) or encoding chemokines, cytotoxic genes which confer toxicity on the cells which are expressing them, for example the herpes simplex type 1 (HSV-1) tk gene, antioncogenes, such as the gene associated with retinoblastoma or p53, or else sequences which are able to inhibit the activity of an oncogene, such as an antisense molecule or a ribozyme which is able to degrade a messenger RNA which is specific for an oncogene.

Cytokines are molecules which are naturally produced following stimulation with an antigen or following an inflammatory reaction (Gillis and Williams, 1998, Curr. Opin. Immunol., 10, 501-503) and whose value within the context of treating certain cancers has been demonstrated, in particular, by Oettger (1991, Curr. Opin. Immunol., 3, 699-705). Leroy et al. (1998, Res. Immunol., 149, 681-684) has reported that, following the intratumor administration of recombinant viral vectors, the production of cytokines at the site of the tumor induces an immune response which leads to the growth of the tumor being inhibited. Nevertheless, although encouraging, this antineoplastic response does not lead to the definitive disappearance of the tumor cells and does not, therefore, make it possible to implement a satisfactory antineoplastic treatment.

For their part, chemokines constitute a subclass of the cytokine family. They are distinguished from other cytokines by their chemoattractive property, in particular during the normal processes of chemotaxis, particularly with regard to attracting cells of the immune system toward the tissues where the inflammation or infection is sited; they are also distinguished by their antiangiogenic properties. Chemokines are proteins which are of small size (from 70 to 80 amino acids) and whose amino acid sequences exhibit only a low level of homology (varying from 10 to 70% depending on the chemokines under consideration). To date, some 50 different chemokines have been identified (see, for example, Zlotnik and Yoshie, 2000, Immunity 12, 121-127, which describes the classification of the chemokines and their respective role in the immune mechanism). Four families have been identified on the basis of the positions of the cysteine residues which they contain. The families α, whose N-terminal end contains 2 cysteines separated by a single amino acid (i.e. the chemokines IL-8, NAP-2 and GCP-2), and ∃ whose N-terminal end contains 2 adjacent cysteines (i.e. the chemokines RANTES, MIP-1 and MCP1), are those which are best characterized (Horuk, R., 1994, Trends Pharmacol. Sci., 15, 159-165; Murphy, 1994, Annu. Rev. Immunol., 12, 593-633).

Antineoplastic approaches which make use of chemokines have already been suggested in the prior art. Thus, the Dilloo et al. group (1996, Nature Medicine 2 (10), 1090-1095) has shown that the antineoplastic immune response of the animal being treated can be stimulated by administering fibroblasts which have been modified ex vivo using retroviral vectors which are coexpressing a specific chemokine, i.e. lymphotactin (Lptn) and interleukin-2 (IL-2). However, this effect is time-limited and only enables the volume of the tumor to be controlled transiently. International application WO 00/74629 describes an approach which is based on combining a chemokine, i.e. MIP-1, with a cytokine (IL-2 or IFNγ). The results obtained following the intratumor administration of adenoviral vectors expressing the two types of polypeptides demonstrate retardation in the proliferation of the tumor and an improvement in survival rate. Nevertheless, although encouraging, this antineoplastic response does not enable complete remission to be achieved in the animals which have been treated.

Incidentally, activation of the T cells is triggered by interaction between the TCR (or TCR/CD3) complex and an antigenic peptide which is presented by the APCs in the context of the major histocompatibility complex. It was well established that naïve CD4+ T cells require 2 signals for inducing an effective immune response: a first signal involving specific recognition of the MHC/peptide by the TCR/CD3 complex and the CD4 coreceptor, and a second signal termed a costimulation signal. While costimulation amplifies and prolongs the primary activation by the TCR, it also inhibits apoptosis of the T cells following activation. The TCR is composed of two functionally distinct units. A heterodimer (αβ or γδ) which is specific for each lymphocyte is required for recognizing the antigen. Engagement of the TCR with its ligand CD3 initiates a cascade of intracellular signals which results in adhesion, proliferation, differentiation into mature cells and an increase in the transcription activity of the genes for cytokines and for cytokine receptors and of the protooncogenes.

Another therapeutic strategy, which combines the advantages of gene therapy and the use of specific antibodies, has also been proposed. The use of gene therapy to produce antibodies, antibody fragments or antibody derivatives, such as chimeric antibodies, in eukaryotic cells is now a standard technique (EP 0 120 694 or EP 0 125 023). For example, international patent application WO 00/24896 describes a method for treating or preventing cancer, with this method being based on expressing antibodies which are specific for the TCR/CD3 complex on the surface of tumor cells with the aim of inducing stimulation of the T cells and an antineoplastic immune response. However, the experimental data show that this approach is effective in certain cancer models, making it possible to attract T lymphocytes and dendritic cells to the site of the tumor and thus induce rejection of the transplanted tumor, they also show that this approach only makes it possible to achieve transient regression in the volume of the tumor, and no complete remission has been obtained in the animals which have been treated.

It is therefore desirable to have available novel compositions which make it possible, in particular, to implement antineoplastic treatments which are effective, which are easy to set up, which make it possible to control the volume of the tumor over an extended period and which increase the survival rate of the treated patients.

The applicant has now identified novel compositions the different constituents of which are selected such that their respective activities have a synergistic effect and that the properties of said constituents are improved. More specifically, these compositions bring about an improvement in the recruitment of the different effectors of the antineoplastic immune response (T cells, NK cells, dendritic cells, macrophages, etc.), thereby making it possible to inhibit or retard proliferation of the target cells and induce their apoptosis (cell death). The present invention offers an advantageous and effective alternative to the approaches of the prior art, in particular for treating or preventing cancer in humans or animals.

In the first place, the present invention relates to a biological material which comprises at least one first and at least one second nucleic acid sequence, said nucleic acid sequences being placed under the control of elements which ensure their expression in a host cell, wherein:

said first nucleic acid sequence encodes all or part of an antibody, characterized in that, when this said antibody or this said antibody part is expressed, it is located on the surface of said host cell and in that said antibody or said antibody part is able to bind to a polypeptide which is present on the surface of a cytotoxic effector cell or of a T helper lymphocyte, said polypeptide being involved in the process of activating such a cell,

said second nucleic acid sequence encodes all or part of a polypeptide which is involved in the stimulation of the immune response and/or in the attraction to the expression site, and in the activation of the cytotoxic effector cells and/or T helper lymphocytes. According to a preferred case, said polypeptide is selected from chemokines and costimulation molecules.

“Nucleic acid sequence” is understood as meaning a natural, isolated or synthetic, linear or circular, double-stranded or single-stranded, DNA and/or RNA fragment which designates a precise sequence of modified or unmodified nucleotides, thereby making it possible to define a fragment or region of a nucleic acid without limiting its size. According to a preferred embodiment, the DNA and/or RNA fragment is a nucleic acid which is selected from the group consisting of a cDNA; a genomic DNA; a plasmid DNA and a messenger RNA.

According to the invention, said “first nucleic acid sequence” encodes, in particular, all or part of a native antibody, or encodes a derivative of such an antibody, as long as said antibody, fragment or antibody derivative is expressed on the surface of the host cell into which a said first nucleic acid sequence has been introduced and as long as said antibody is able to bind to a polypeptide which is present on the surface of a cytotoxic effector cell or of a T helper lymphocyte and which is involved in the process of activating such a cell. More specifically, antibody “fragment” is understood as meaning the F(ab)₂, Fab′, Fab, Fv, sFv (Blazar et al., 1997, J. of Immunology, 159, 5821-5833; Bird et al., 1988, Science, 242, 423-426) and dAbs (Ward et al., 1989, Nature, 341, 544) fragments of a native antibody which may be of polyclonal or monoclonal origin (see, for example, Monoclonal Antibodies: A manual of Techniques and Applications II. Zola (CRC Press, 1988) and Monoclonal Hybridoma Antibodies: Techniques and Applications, J. Hurrell (CRC Press, 1982)). Incidentally, a general review of the techniques relating to synthesizing antibody fragments which retain the binding specificity of the native antibody is available in Winter et al. (1991, Nature, 349, 293-299). An antibody “derivative” is understood as meaning, for example, a chimeric derivative of such an antibody (see, for example, the chimeras the mouse/man anti-CD3 antibodies in Arakawa et al., 1996, J. Biochem., 120, 657-662 or the immunotoxins such as sFv-toxin of Chaudary et al., 1989, Nature, 339, 394-397).

“Antibody which is expressed on the surface of a host cell” is understood as meaning an antibody of which at least the functional region, which is able to recognize and bind to its specific antigen, is expressed on the surface of the host cells in order to permit said recognition and binding. More specifically, the antibodies which are used within the context of the present invention consist of fusion polypeptides which comprise at least the amino acids which define said functional region and a peptide which is able to confer, to the antibody, a transmembrane localization in the host cell. The term “transmembrane localization” refers to anchorage within the membrane lipid double layer of the host cell or on the external surface of this bilayer. The nucleic acid sequences encoding a large number of transmembrane peptides are described in the literature. Advantageously, said transmembrane peptide is isolated or derived from a glycoprotein, a lipoprotein or a membrane receptor. According to a preferred embodiment of the invention, the transmembrane peptide is isolated from a glycoprotein such as measles virus glycoprotein F (European patent EP 0 305 229), CD4 (Weijtens et al., 1998, Gene Therapy, 5, 1195-1203), HIV virus gp160 (Polydefkis et al., 1990, J. Exp. Med., 171, 875-887), Fc (see below) and, especially, rabies virus glycoprotein (French patent application FR 97 09152).

According to an altogether advantageous embodiment, the first nucleic acid sequence contains a gene encoding the heavy chain of said antibody fused to the nucleic acid sequence encoding a transmembrane peptide as defined above. In addition, the first nucleic acid sequence can contain a gene encoding the light chain of said antibody. While expression of the heavy and light chains can be controlled by independent regulatory elements, it is also possible to use common elements (bicistronic cassette) and, where appropriate, to re-initiate translation of the second cistron (for example the sequences encoding the light chain) by means of an IRES (WO 98/49334).

According to the invention, the antibody which is expressed on the surface of the host cells is able to bind to a polypeptide which is present on the surface of a cytotoxic effector cell or of a T helper lymphocyte, in particular a CD4 T helper lymphocyte, and which is involved in the process of activating such a cell, more specifically to a receptor which is directly involved in such a process. As has been described previously, this phenomenon of activating cytotoxic effector cells or T helper lymphocytes is a crucial element in the cell-mediated immune reaction. However, it should be noted that, within the context of implementing the present invention, it is not essential that the activation process takes place after binding by the antibody which is expressed on the surface of the host cells. Thus, in accordance with the invention, this antibody can also bind to the polypeptides which are present on cytotoxic effector cells or helper lymphocytes which are already activated.

“Cytotoxic effector cell” is understood as meaning macrophages, cytotoxic T lymphocytes (CTLs) and killer cells (NKs) as well as their derived cells such as LAK cells (Versteeg, 1992, Immunology Today, 13, 244-247; Brittende et al., 1996, Cancer, 77, 1226-1243; Poplack et al., 1976, Blood, 48, 809-816). “T helper lymphocyte” is understood as meaning, in particular, the CD4 cells which, following activation, make possible the secretion of factors for activating the effector cells of the immune response (see above) . The polypeptides, in particular the receptors, which are expressed on the surface of these cells, and which are involved in the activation of these cells, consist, in particular, of all or part of the TCR complex, more especially TCR-α, TCR-β, or CD3, all or part of the CD8, CD4, CD28, LFA-1, 4-1BB (Mclero et al., 1998, Eur. J. Immunol., 28, 1116-1121), CD47, CD2, CD1, CD9, CD45, CD30 and CD40 complexes, all or part of the cytokines receptors (Finke et al., 1998, Gene Therapy, 5, 31-39), such as IL-7, IL-4, IL-2, IL-12, IL-15, IL-18, IL-21 or GM-CSF, all or part of the NK cell receptor complex, such as Vα14NKT (Kawano et al., 1998, Immunology, 95, 5690-5693), NKAR, Nkp46 (Pessino et al., 1998, J. Exp. Med., 188, 953-960) or Nkp44, or all or part of the macrophage receptors, such as the Fc receptor (Deo et al., 1997, Immunology Today, 18, 127-135).

According to a particular embodiment, it is also possible to envisage genetically modifying, particularly in vivo, the cytotoxic effector cells or the T helper lymphocytes such that they express, at their surface, a polypeptide, which is not naturally expressed by these cells and which is capable of inducing the process of activating these cells, by means of introducing, into these cells, nucleic acid sequences which encompass the gene which encodes such a polypeptide. In accordance with the present invention, it is then possible to select a nucleic acid sequence which encodes all or part of an antibody which is capable of being expressed on the surface of the target cells of the patient to be treated, with said antibody being capable of binding to such a polypeptide which is not naturally expressed by these cytotoxic effector cells or T helper lymphocytes.

More especially, the present invention is based on the possibility of cloning the genes encoding all or part of an antibody and expressing said antibody in cells after said genes have been transferred into said cells using conventional expression vectors (see below) . The literature suggests a large number of examples of genes which encode antibodies which are able to react with such polypeptides or receptors. It is within the capacity of the skilled person to obtain the nucleic acid sequences which encode such antibodies. Mention may be made, for example, of the genes encoding the light and heavy chains of the antibody YTH 12.5 (anti-CD3) (Routledge et al., 1991, Eur. J. Immunol., 21, 2717-2725), and anti-CD3 in accordance with Arakawa et al. (1996, J. Biochem., 120, 657-662). The nucleic acid sequences for such antibodies are readily identifiable from the databases which are commonly employed by the skilled person.

By way of illustration, it is also possible to cite the following hybridomas as being particularly appropriate for isolating the first nucleic acid sequence as defined above:

hybridoma TR310 (rat anti-murine Vβ7 (IgG2b); ATCC HB-219; I. L. Weissman; murine myeloma/rat splenocyte fusion);

hybridoma H57-597 (hamster anti-murine TCRαβ (IgG); ATCC HB-218; Kubo et al., 1989, J. Immunology, 142, 2736-2742; murine myeloma/hamster splenocyte fusion);

hybridoma KT3 (rat anti-murine CD3ε (IgG2a); Tomonari et al., 1988, Immunogenetics, 28, 455-458; murine myeloma/rat splenocyte fusion).

The nucleic acid sequences encoding the heavy and/or light chains of these different antibodies can be obtained using the methods which are conventional in the field, in particular amplification (PCR, RT-PCR, etc.) , cloning using specific oligonucleotides or the techniques which make use of cDNA libraries (Maniatis et al., 1982, Molecular cloning. A laboratory manual. C.S.H. Laboratory, Cold Spring Harbor, N.Y.) from hybridomas which are available from accredited collections (such as the ATCC) and which secrete antibodies which are specific for polypeptides which are present on the surface of cytotoxic effector cells or T helper lymphocytes and which are involved in the process of activating such cells (for example a hybridoma which excretes immunoglobulins Gγ2b+κ, which are directed against the TCR receptors, or one of those cited above) These sequences which have thus been cloned are then available for being cloned into vectors. As previously mentioned, the nucleic acid sequence encoding the heavy chain of the antibody is, according to a preferred embodiment of the invention, fused to the nucleic acid sequence encoding a transmembrane peptide such as the rabies glycoprotein. The techniques of molecular biology are fully described in the French patent application FR 97 09152).

Within the context of the present invention, the “second nucleic acid sequence” encodes all or part of a polypeptide which is involved in stimulating the immune response and/or attracting cytotoxic effector cells and/or T helper lymphocytes to the site of expression and activating them. According to a particularly preferred embodiment, said polypeptide is selected from chemokines and costimulation molecules.

Within the meaning of the present invention, stimulating the immune response makes it possible to induce or activate an immune response which is specifically directed against a cell, in particular a tumor cell or a cell which is infected with a virus, or to inhibit the growth and/or division of such a cell. The attraction to the expression site, and the activation, of the cytotoxic effector cells or T helper lymphocytes can be defined by the capacity to increase the migration of the immune cells toward the site at which a chemokine is expressed for the purpose of developing or prolonging an immune response.

Within the meaning of the present invention, the term “chemokine” can be defined as a cytokine which has a chemoattractive effect (ability to attract immune cells to the site at which said chemokine is expressed). The chemokine which is used within the context of the present invention preferably possesses one or more supplementary functions, in particular activation of the immune cells, making it possible to stimulate the immune response, and/or an antiangiogenic function, making it possible to inhibit vascularization within a tumor. The chemoattractive activity of a given polypeptide, in particular a polypeptide derived from the chemokine MIP, on cells involved in the immune reactions (such as eosinophils, T lymphocytes, monocytes or neutrophils) can be assessed using a chemotaxis test (Maghazachi, 1993, Nature Immunity, 12, 57). Similarly, since this type of chemokine inhibits the proliferation of hematopoietic precursors, it is possible to assess such a property in vitro using the method described by Graham et al. (1992, Growth Factors, 7, 151).

Within the meaning of the present invention, the term “costimulation molecules” refers to a molecule which makes it possible to amplify an immune response and/or prolong the state of activation of an immune cell or of a cell which is involved in an immune response, in particular by stabilizing the TCR complex after the peptide linked to the MHC has been recognized. The costimulation activity can be assessed using conventional techniques such as those described in the example section of the present application (for example by measuring the proliferation of naïve splenocytes following the incorporation of ³H thymidine, as described in item 5 in example 2).

Within the context of the present invention, it is possible to use a second nucleic acid sequence which encodes the whole of said polypeptide in question or only a part of this polypeptide, or encodes a derived or mutated polypeptide, as long as the function and the properties, in terms of stimulating the immune response, and of attracting cytotoxic effector cells or T helper lymphocytes to the expression site and/or activating them, are retained. As previously mentioned, the term “mutation” refers to the deletion and/or replacement and/or addition of one or more nucleotides, or to any combination of this type of mutation. In the same way, it is possible to envisage to use a sequence which encodes a hybrid polypeptide which is derived from the fusion of sequences of differing origin (for example encoding two different chemokines).

Chemokines which can be used within the context of the present invention and which may be mentioned are RANTES (GenBank M21121), MIG (GenBank M34815), IL-8 (GenBank M28130), MCP-1 (GenBank X14788), BRAK (Frederick et al., 2000, Am. J. Pathol., 156, 1937-50; Sleeman et al., 2000, Int. Immunol., 12, 677-689) and type 1 MIP (MIP-1) or type 2 MIP (MIP-2). Preference is given to an MIP-1 chemokine, with this chemokine being more especially selected from the group consisting of the chemokines MIP-1α and MIP-1β, whose properties have been demonstrated by Wolpe et al. (1988, J. Exp. Med. 167, 570-581). MIP-1α is produced by T lymphocytes and monocytes. It is responsible for the chemoattraction of eosinophils and T lymphocytes during infections of the respiratory tract; it is also responsible for the chemoattraction of monocytes and neutrophils during the course of rheumatoid arthritis, inflammations of the digestive system or meningites of bacterial origin. Furthermore, it inhibits the proliferation of hematopoietic precursors. The MIP-1α nucleotide and amino acid sequences are described in Obaru et al. (1986, J. Biochem. 99, 885-894), the content of which is hereby incorporated by reference into the present application. MIP-1β, the nucleotide and amino acid sequences of which are described in Brown et al. (1989, J. Immunol. 142, 679-88, the content of which is hereby incorporated by reference into the present application), is also produced by the T lymphocytes and the monocytes. It exerts its chemoattractive properties on monocytes and neutrophils in cases of osteoarthritis and bacterial meningitis. Like MIP-1α, it inhibits the proliferation of hematopoietic precursors. Natural variants of said MIP-1α and MIP-1β proteins exist, with these variants being known to the skilled person and being termed, for example, GOS19, LD78, pAT464, (mouse) TY5 or (mouse) SIS α, in the case of MIP-1α, and pAT744, Act-2, G-26, (mouse) H-400 or (mouse) hSIS γ, in the case of MIP-1β. In the particular case of MIP-1β, preference will be given to selecting the sequence corresponding to Act-2 (Lipes et al., 1988, Proc. Natl. Acad. Sci. USA 85, 9704-9708, the content of which is hereby incorporated by reference into this present document).

Of the costimulation molecules which can be used within the context of the present invention, preference is given to employing those which act on the costimulation of the T cells, in particular B7-1 (GenBank XM002948) and B7-H1 (GenBank AF177937). Costimulating the T cells decreases their apoptosis, as engendered by the first activation signal (AICD; activation-induced cell death), by inducing the expression of anti-apoptotic factors such as Bcl-2, c-FLIP and Bcl-xL and by reducing the activity of the Fas apoptotic complex and procaspase-8. Costimulation also promotes differentiation of activated T cells into memory cells. The present invention also relates to any other molecule which enables said costimulation of the T cells to be effected.

According to a particularly preferred embodiment, the second nucleic acid sequence encodes a polypeptide which is secreted by the host cell. The means for obtaining secretion of a polypeptide from the host cell are known to the skilled person. It is possible, in particular, to make use of a signal peptide, which can be that which is naturally used by the polypeptide in question (homologous) or which can be heterologous to this polypeptide and isolated from a protein which is secreted naturally. The prior art discloses a large number of signal peptides which are functional in eukaryotic cells, in particular mammalian cells.

According to another variant of the present invention which is also preferred, the biological material according to the present invention additionally includes at least one third nucleic acid sequence which encodes all or part of a polypeptide having a cytotoxic activity.

“Polypeptide having a cytotoxic activity” is understood as meaning any polypeptide which is able to inhibit the growth and/or division of a host cell, in particular a tumor cell (the cytotoxic activity is then termed antineoplastic activity) or an infected cell (the cytotoxic activity is then termed antiviral activity, antiparasitic activity or antibacterial activity, depending on the pathogenic agent which is infecting the cell). According to a preferred embodiment, the cytotoxic activity is expressed by the death of said cell. According to a particular embodiment, it would also be possible to use the biological material according to the present invention in disease situations which are associated with cell proliferation, for example restenosis phenomena. The cytotoxic activity of a given polypeptide, in particular with regard to tumor cells, can be assessed in vitro, by measuring cell survival, either using short-term viability tests (such as the tryptan blue or MTT test) or using clonogenic survival tests (formation of colonies) (Brown and Wouters, 1999, Cancer Research 59, 1391-1399), or in vivo, by measuring tumor growth (size and/or volume) in an appropriate animal model (Ovejera and Houchens, 1981, Semin. Oncol. 8, 386-393).

Said polypeptide having a cytotoxic activity is advantageously selected from cytokines, proteins encoded by the suicide genes and antiangiogenic protein factors.

When said polypeptide is a cytokine, it is preferably a cytokine which is selected from interferons α, β and γ, interleukins, in particular IL-2, IL-4, IL-6, IL-7, IL-10, IL-12, IL-15, IL-18 or IL-21, tumor necrosis factors (TNFs) and colony stimulating factors (GM-CSF, C-CSF, M-CSF, etc.).

According to a preferred embodiment, said cytokine is selected from interleukin-2 (IL-2) and interleukin-12 (IL-12) . Interleukin-2 is responsible, in particular, for the proliferation of activated T lymphocytes and the multiplication and activation of cells of the immune system (for the nucleic acid sequence, see, in particular, FR 85 09480). Interleukin-12 was identified and isolated in macrophages as being a factor for stimulating cytotoxic T cells and NK cells, with the factor inducing differentiation of CD4⁺ T lymphocytes by participating in the homeostasis of Th1 and Th2 populations. Two cell types (NK cells and NKT cells) are involved in the antineoplastic activity mediated by IL-12, depending on the dose employed and on the scheme used for injecting it.

According to the second variant, the polypeptide which is produced by expressing a suicide gene possesses at least one enzymic activity selected from thymidine kinase activity, purine nucleoside phosphorylase activity, guanine or uracil or orotate phosphoribosyl transferase activity and cytosine deaminase activity. While these polypeptides are not toxic as such, they exhibit catalytic enzymic properties which are able to transform an inactive substance (prodrug), for example a nucleoside or a nucleoside analog, into a substance which is highly toxic for the cell, for example a modified nucleoside which can be incorporated into the DNA or RNA chains by elongation resulting, in particular, in the inhibition of cell division or in cell dysfunction, leading to the death of the cell which contains such polypeptides. The genes encoding these polypeptides are termed “suicide genes”. A large number of suicide gene/prodrug pairs are currently available. Those which may more specifically be mentioned are type 1 herpes simplex virus thymidine kinase (HSV-1 TK) and acyclovir or ganciclovir (GCV) (Caruso et al., 1993, Proc. Natl. Acad. Sci., USA 90, 7024-7028; Culver et al., 1992, Science, 256, 1550-1552; Ram et al., 1997, Nat. Med., 3, 1354-1361); and cytosine deaminase (CDase) and 5-fluorocytosine (5-FC). The Saccharomyces cerevisiae (S. cerevisiae) FCY1 gene and the E. coli codA gene, respectively encoding the CDases in these two organisms, are known and their sequences have been published (EP 0 402 108; Erbs et al., 1997, Curr. Genet. 31, 1-6; WO 93/01281). The CDase is advantageously employed in combination with the enzyme uracil phosphoribosyl transferase (UPRTase), which has the property of converting the 5-FU which is produced by the action of the CDase into the highly toxic 5-FUMP. The upp and FUR1 genes, respectively encoding the S. cerevisiae UPRTase and the E. coli UPRTase, have been cloned and sequenced (Andersen et al., 1992, Eur. J. Biochem., 204, 51-56; Kern et al., 1990, Gene 88, 149-157). It is possible to use mutants of these suicide gene expression products, such as those described in patent applications WO 96/16183 and WO 99/54481.

According to a preferred embodiment, the biological material according to the invention comprises a first nucleic acid sequence, encoding the antibody KT3, which is expressed in transmembrane form and which is able to bind the TCR complex, and a second nucleic acid sequence, encoding the chemokine MIP-1β. Another preferred biological material comprises a first nucleic acid sequence, encoding the antibody KT3, which is expressed in transmembrane form and which is able to bind the TCR complex, and a second nucleic acid sequence, encoding the chemokine BRAK. According to an even more preferred embodiment, the biological material additionally includes at least a third nucleic acid sequence encoding IL-2 or IL-12.

The different nucleic acid sequences which are used within the context of the present invention are, of course, placed under the control of elements which ensure that they are expressed in the host cell. While preference is given to each nucleic acid sequence being able to be directed by elements (which are identical or different, homologous or heterologous with regard to the nucleic acid sequence in question and constitutive or inducible) which are specific to itself, it is also possible to use communal elements for directing the expression of two, if not to say three, nucleic acid sequences employed within the context of the present invention. In this case, it is possible either to use fused sequences or to use IRESs for reinitiating translation of the cistrons.

“Elements which ensure expression” is understood as meaning the elements which are required for ensuring that the nucleic acid sequence is expressed after it has been transferred into a target cell. The elements are, in particular, promoter sequences and/or regulatory sequences which are effective in said cell. The promoter which is used can be a ubiquitous or tissue-specific viral or cellular promoter or else a synthetic promoter. It can, of course, be modified as compared with the native promoter sequence in order to include or delete one or more restriction sites or else delete negative sequences which reduce transcription levels, etc.

By way of example, mention may be made of the viral promoters, in particular the promoters of the RSV (Rous sarcoma virus), SV40 (simian virus) and CMV (cytomegalovirus) viruses, the early (E1a, E3, etc.) and late (MLP, standing for major late promoter) adenoviral promoters, the retroviral LTRs (like that of the MLV, standing for murine leukemia virus, virus) and the HSV-1 TK promoter. The promoters which can be used within the context of a poxvirus vector can be selected from the vaccinia virus promoters 7.5K, H5R, TK, p28, p11 and K1L as well as hybrid early-late promoters and synthetic promoters (Chakrabarti et al., 1997, Biotechniques 23, 1094-1097; Hammond et al., 1997, J. Virological Methods 66, 135-138; Kumar and Boyle, 1990, Virology 179, 151-158).

In addition, it is possible to select a promoter sequence which is specific for a given cell type or which can be activated under defined conditions. The literature provides a large amount of information relating to such promoter sequences. Mention may be made, more especially, of the promoters of the MT (metallothionein; McIvor et al., 1987, Mol. Cell Biol. 7, 838-848) genes and those encoding α-1 antitrypsin, CFTR, pulmonary surfactant, immunoglobulins, muscle creatine kinase, β-actin, SRα, SM22 protein (Moessler et al., 1996, Development, 122, 2415-2425) and desmin (WO 96/26284). It is also possible to use a promoter which can be activated in dividing cells, for example a promoter which governs the transcription of genes which are overexpressed in tumor cells. In this regard, mention may be made of the promoters of the genes which encode MUC-1, which is overexpressed in breast cancer and prostate cancer (Chen et al., 1995, J. Clin. Invest. 96, 2775-2782), CEA (carcinoma embryonic antigen), which is overexpressed in cancer of the colon (Schrewe et al., 1990, Mol. Cell. Biol., 10, 2738-2748), ERB-2, which is overexpressed in breast and pancreas cancers (Harris et al., 1994, Gene Therapy 1, 170-175) and α-fetoprotein, which is overexpressed in liver cancers (Kanai et al., 1997, Cancer Res., 57, 461-465).

Furthermore, the elements ensuring expression can also include what are termed enhancer sequences and LCR (standing for locus control region) sequences, which improve or stabilize expression of the nucleic acid sequence in the host cell or confer tissue-specific expression. The elements ensuring expression of said nucleic acid sequences can also comprise sequences which are required for intracellular transport, for replication and/or for integration, for transcription or for translation. The literature provides a large amount of information relating to such regulatory elements. Similarly, the nucleic acid sequences which are used within the context of the present invention can comprise “neutral” sequences, or introns, which do not harm transcription and which are spliced out before the translation step. These sequences, and their uses, are described in the literature (WO 94/29471). In addition, the nucleic acids which can be used in accordance with the present invention can also be nucleic acids which are modified such that they are unable to integrate into the genome of the target cell or nucleic acids which are stabilized using agents, such as spermine, which do not, as such, have any effect on the efficacy of the transfection.

Said nucleic acid sequences can be controlled by elements which are identical or different, and can be located with respect to each other such that they are contiguous or at a distance and in the same direction or in opposite directions, as long as their expression in the host cell is not affected.

According to a first embodiment of the invention, the biological material according to the invention is in the form of DNA or RNA which is naked, that is to say free of any compound which might facilitate its introduction into the cells (nucleic acid sequence transfer).

However, in order to promote their introduction into the host cells, and thus obtain host cells which are genetically modified, said first and second (and, optionally, third) nucleic acid sequences of the biological material according to the invention can be comprised in at least one vector enabling them to be transferred into the host cell. As already mentioned, they can be contained in one and the same vector or, in accordance with a preferred embodiment, in independent vectors which enable them to be transferred into the host cell.

According to the invention, such a vector can, in this context, be a plasmid. There is a vast choice of plasmids which can be used within the context of the present invention. The plasmids can be cloning vectors and/or expression vectors. They are well known to the skilled person and a number of them are available commercially. However, it is also possible to construct them or modify them using the techniques of genetic manipulation. Those which may be mentioned, by way of example, are the plasmids which are derived from pBR322 (Gibco BRL), pUC (Gibco BRL), pBluescript (Stratagene), pREP4, pCEP4 (Invitrogen) or else p Poly (Lathe et al., 1987, Gene, 57, 193-201). A plasmid which is used within the context of the present invention preferably contains an origin of replication which ensures that replication is initiated in a producing cell and/or a host cell (for example, the ColE1 origin will be selected in the case of a plasmid which is intended to be produced in E. coli while the oriP/EBNA1 system will be selected if the plasmid is required to be self-replicating in a mammalian host cell, Lupton and Levine, 1985, Mol. Cell. Biol., 5, 2533-2542; Yates et al., 1985, Nature 313, 812-815). The plasmid can also include a selection gene which enables the transfected cells to be selected or identified (complementation of an auxotrophic mutation, gene encoding resistance to an antibiotic, etc.). The plasmid can, of course, contain additional elements which improve its maintenance and/or stability in a given cell (cer sequence which promotes maintenance of a plasmid in monomeric form (Summers and Sherrat, 1984, Cell 36, 1097-1103, sequences for integration into the cell genome).

The vectors which are used within the context of the present invention are preferably viral vectors, for example vectors which are selected from adenoviral, retroviral and poxviral vectors, in particular vectors which are derived from the vaccinia virus (Goebel et al., 1990, Virol., 179, 247-266 and 517-563; Johnson et al., 1993, Virol., 196, 381-401), from modified Ankara virus (MVA) (Antoine et al., 1998, Virol., 244, 365-396) or from canarypox virus, or vectors which are derived from herpesvirus, alphavirus, foamyvirus or adenovirus-associated virus. Preference will be given to using a vector which does not replicate and which does not integrate. However, it should be noted here that, within the context of implementing the present invention, the nature of the vector is of little importance since the transfer only takes place in order to enable the (first, second and, possibly, third) nucleic acid sequences to be transferred into the interior of the host cells. Such transfer models are used widely in the literature.

The basic technology for inserting a nucleic acid sequence, and its regulatory elements, into a poxvirus genome is described in a large number of documents available to the skilled person (Piccini et al., 1987, Methods of Enzymology, 153, 545-563; U.S. Pat. No. 4,769,330; U.S. Pat. No. 4,772,848; U.S. Pat. No. 4,603,112; U.S. Pat. No. 5,100,587 and U.S. Pat. No. 5,179,993). This technique is based on homologous recombination between sequences which are common to the viral genome and to a transfer plasmid which is carrying the nucleic acid sequence to be transferred. The insertion site in the poxvirus genome is preferably a nonessential locus. Such nonessential loci are located in noncoding intergene regions or within a gene whose deletion or lack of function does not affect, or only has a slight effect on, viral growth, replication or infection. It is also possible to envisage insertion into an essential locus on condition that the affected function is complemented in trans, using, for example, a complementation line or a helper virus which carries the sequences encoding the deficient function, while the viral particles are being produced. In the case of a vaccinia virus of the Copenhagen strain, a preferred insertion site is located within the thymidine kinase (TK) gene (Hruby et al., 1983, Proc. Natl. Acad. Sci. USA, 80, 3411-3415; Weir et al., 1983, J. Virol., 46, 530-537). In the case of an MVA vector, the nucleic acid sequence is inserted into one of the deletions I to VII, preferably into deletion II or deletion III (Meyer et al., 1991, J. Gen. Virol., 72, 1031-1038; Sutter et al., 1994, Vaccine, 12, 1032-1040). The conditions for constructing a recombinant vaccinia virus are known to the skilled person (see, for example, EP 83 286 and EP 206 920, in the case of vaccinia virus, and Mayr et al., 1975, Infection, 3, 6-14 and Sutter and Moss, 1992, Proc. Natl. Acad. Sci. USA, 89, 10847-10851 in the case of MVA).

Retroviruses have the property of in the main infecting, and integrating into, dividing cells and, in this regard, are particularly appropriate for the cancer application. In general, a recombinant retrovirus according to the invention contains LTR sequences, an encapsidation region and at least one of the nucleic acid sequences used within the context of the invention, with this sequence being placed under the control of the retroviral LTR or of an internal promoter such as those described below. The recombinant retrovirus can be derived from a retrovirus of any origin (murine, primate, feline, human, etc.) and, in particular, from the MoMuLV (Moloney murine leukemia virus; Gilboa et al., 1988, Adv. Exp. Med. Biol., 241, 29), MVS (murine sarcoma virus) or Friend murine retrovirus (Fb29; WO 95/01447). The recombinant retrovirus is propagated in an encapsidation cell line which is able to supply in trans the viral polypeptides gag, pol and/or env, which are required for constituting a viral particle. Such cell lines are described in the literature (PA317, Psi CRIP GP+Am-12, etc.). The retroviral vector according to the invention can contain modifications, in particular at the level of the LTRs (replacement of the promoter region with a eukaryotic promoter) or of the encapsidation region (replacement with a heterologous encapsidation region, for example of the VL30 type) (see French applications 94 08300 and 97 05203 and U.S. Pat. No. 5,747,323).

It will also be possible to use an adenoviral vector. While this can be a vector which is not defective, it preferably replicates conditionally (CRAd; Heise and Kim, 2000, J. Clin. Invest., 105, 847851; Alemany et al., 2000, Nature Biotechnology, 18, 723-727; Hernandes-Alcoceba et al., 2000, Human Gene Ther., 11, 2009-2024) or is defective for replication, that is to say lacks all or part of at least one region which is essential for replication and which is selected from the regions E1, E2 and E4. A deletion of the E1 region is preferred. However, it can be combined with (an)other modification(s)/deletion(s) which, in particular, affect(s) all or part of the regions E2, E4 and/or L1-L5 as long as the essential defective functions are complemented in trans with a complementation line and/or a helper virus in order to ensure production of the viral particles of interest. In this regard, it is possible to use second generation vectors of the state of the art (see, for example, International applications WO 94/28152 and WO 97/04119). By way of illustration, deletion of the majority of the E1 region and of all or part of the E4 transcription unit is very particularly advantageous (EP 974 668). With the aim of increasing its cloning capacity, the adenoviral vector can additionally lack all or part of the nonessential E3 region. According to another alternative, it is possible to use a minimal adenoviral vector which retains the sequences which are essential for encapsidation, namely the 5′ and 3′ ITRs (inverted terminal repeats) and the encapsidation region. Furthermore, the origin of the adenoviral vector according to the invention can vary, both as regards species and as regards serotype. The vector can be derived from the genome of an adenovirus of human or animal (canine, avian, bovine, murine, ovine, porcine, simian, etc.) origin or else from a hybrid comprising adenoviral genome fragments of at least two different origins. More specific mention may be made of the adenoviruses CAV-1 or CAV-2, of canine origin, DAV, of avian origin, or else type 3 Bad, of bovine origin (Zakharchuk et al., Arch. Virol., 1993, 128:171-176; Spibey and Cavanagh, J. Gen. Virol., 1989, 70:165-172; Jouvenne et al., Gene, 1987, 60:21-28; Mittal et al., J. Gen. Virol., 1995, 76:93-102). However, preference will be given to an adenoviral vector of human origin which preferably derives from a serotype C adenovirus, particularly type 2 or type 5. An adenoviral vector according to the present invention can be generated in vitro, by ligation or homologous recombination in Escherichia coli (E. coli) (see, for example, International application WO 96/17070) or else by recombination in a complementation cell line (see, for example, Graham and Prevect, 1991, in Methods in Molecular Biology, vol. 7, p 109-128; Ed.: E. J. Murey, The Human Press Inc.).

According to a preferred embodiment, the first nucleic acid sequence is carried by a poxvirus vector which is derived from the modified Ankara virus (MAV) strain of the vaccinia virus and the second nucleic acid sequence is carried by an adenoviral vector. When the biological material according to the invention comprises a third nucleic acid sequence, this latter sequence is preferably carried by an adenoviral vector.

It is pointed out that, within the context of the present invention, the term “biological material” comprises the viral vector (recombinant genome) and the infectious viral particles which contain said viral vector. Such a viral particle can be generated from a viral vector using any technique which is conventional in the relevant field. It is propagated, in particular, in a complementation cell which is adapted for the deficiencies of said vector. In the case of an adenoviral vector, use will be made, for example, of a complementation cell line such as that described in application WO 94/28152, of cell line 293, which was established from human embryonic kidney cells and which efficiently complements the E1 function (Graham et al., 1977, J. Gen. Virol. 36, 59-72), of cell line A549-E1 (Imler et al., 1996, Gene Therapy 3, 75-84) or of a cell line which enables double complementation to take place (Yeh et al., 1996, J. Virol. 70, 559-565; Krougliak and Graham, 1995, Human Gene Therapy 6, 1575-1586; Wang et al., 1995 Gene Therapy 2, 775-783; International application WO 97/04119). It is also possible to use helper viruses in order to complement at least some of the defective functions. A complementation cell is understood as meaning a cell which is able to provide, in trans, the early and/or late factors which are required to encapsidate the viral genome in a viral capsid in order to generate a viral particle which contains the recombinant vector. Said cell may not, on its own, complement all the defective functions of the vector and, in that case, can be transfected/transduced with a helper vector/virus which supplies the complementary functions. The viral particles containing poxvirus vectors are prepared by infecting permissive cells (for example chick embryo primary fibroblasts) using the techniques of the art, which techniques are detailed widely in the documents which are cited in the poxvirus field.

The invention also relates to a method for preparing a viral particle, according to which:

(i) a biological material according to the invention is introduced into a cell, in particular a complementation cell which is. able to complement said vector in trans so as to obtain a said transfected cell,

(ii) said transfected cell is cultured under conditions which are appropriate for enabling said viral particle to be produced, and

(iii)said viral particle is recovered from the cell culture.

Naturally, the viral particle can be recovered either from the culture supernatant or, equally well, from the cells. One of the methods which is currently employed consists in lyzing the cells (chemical lysis, freezing/thawing, osmotic shock, mechanical shock, sonication, etc.) in order to collect the virions in the lysis supernatant. These latter can be amplified and purified using the techniques of the art (chromatographic method, ultracentrifugation, in particular through a cesium chloride gradient, etc.).

According to one variant, the vector which is used in accordance with the invention can be a nonviral vector such as, for example, a vector consisting of at least one said nucleic acid sequence or a plasmid vector as defined above, which vector is complexed or conjugated with at least one carrier substance or molecule which is selected from the group consisting of a cationic amphiphilic compound, in particular a cationic lipid, a cationic or neutral polymer, a protic polar compound which is selected, in particular, from propylene glycol, polyethylene glycol, glycerol, ethanol or 1-methyl-L-2-pyrrolidone or their derivatives, and an aprotic polar compound which is selected, in particular, from dimethyl sulfoxide (DMSO), diethyl sulfoxide, di-n-propyl sulfoxide, dimethylsulfone, sulfolane, dimethylformamide, dimethylacetamide, tetramethylurea or acetonitrile or their derivatives.

Broadly speaking, cationic lipids have a high affinity for nucleic acids and have the ability to interact with the cell membrane (Felgner et al., 1989, Nature, 337, 387-388). The cationic lipids which are very particularly suitable for implementing the present invention include, in particular, DOTMA (Felgner et al., 1987, Proc. Natl. Acad. Sci. USA, 84, 7413-7417), DOGS or Transfectam™ (Behr et al., 1989, Proc. Natl. Acad. Sci. USA, 86, 6982-6986), DMRIE or DORIE (Felgner et al., 1993, Methods, 5, 67-75), DC-CHOL (Gao and Huang, 1991, BBRC, 179, 280-285), DOTAP™ (McLachlan et al., 1995, Gene Therapy, 2, 674-622), Lipofectamine™ and glycerolipid compounds (see, for example, EP 901 463 and WO 98/37916).

The polymers which are suitable for implementing the invention are preferably cationic polymers, such as polyamidoamine (Haensler and Szoka, 1993, Bioconjugate Chem., 4, 372-379), dendritic polymers (WO 95/24221), polyethyleneimine or polypropyleneimine (WO 96/02655), polylysine (U.S. Pat. No. 5,595,897 or FR 2 719 316), chitosan (U.S. Pat. No. 5,744,166) or DEAE dextran (Lopata et al., 1984, Nucleic Acid Res., 12, 5707-5717).

Furthermore, the vectors which are used within the context of the present invention can additionally comprise targeting elements which enable the transfer of said nucleic acid sequences to be directed toward certain cell types or certain specific tissues (tumor cells, cells of the pulmonary epithelium, hematopoietic cell, muscle cell, nerve cell, etc.). The targeting elements can also enable the transfer of an active substance to be directed toward certain preferred intracellular compartments such as the nucleus and the mitochondria. The elements can also be elements which facilitate penetration into the interior of the cell or lysis of the endosomes. Such targeting elements are widely described in the literature. They can, for example, in whole or in part, be lectins, peptides, in particular the peptide JTS-1 (see patent application WO 94/40958), oligonucleotides, lipids, hormones, vitamins, antigens, antibodies, ligands which are specific for membrane receptors, ligands which are able to react with an antiligand, fusogenic peptides or nuclear localization peptides, or a combination of these compounds. In particular, the targeting elements can be galactosyl residues which make it possible to target the asialoglycoprotein receptor on the surface of hepatic cells, or ligands which are able to interact with receptors such as growth factor receptors or receptors for cytokines, lectins or adhesion proteins; the targeting element can also be an antibody fragment such as the Fab fragment, an INF-7 fusogenic peptide which is derived from the HA-2 subunit of influenza virus hemagglutinin (Plank et al., 1994, J. Biol. Chem., 269, 12918-12924), or a nuclear localization signal which is derived from the SV40 virus T antigen or from the Epstein Barr virus EBNA-1 protein.

The mammalian cells which the invention intends to eliminate, or whose growth the invention intends to limit, and which may be mentioned more specifically are tumor cells and cells which are infected with a viral, parasitic or bacterial pathogenic agent. According to the invention, the expression, on the surface of the cells, of all or part of an antibody which is able to bind to a polypeptide, which is present on the surface of a cytotoxic effector cell or of a T helper lymphocyte and which is involved in the process of activating such a cell, makes it possible to direct the cytotoxic immune response toward a given target and, more specifically, to direct this response toward a tumor or a focus of infection.

The examples of viral pathogens which may be mentioned are the viruses HIV, EBV and CMV, hepatitis B and C viruses and papillomaviruses. Examples of parasitic pathogenic agents which may be mentioned are Leishmania lesmaniae and Plasmodium falciparum.

The invention also relates to a host cell which comprises a biological material according to the invention. The host cell is preferably a mammalian tumor cell or a mammalian cell which is infected with a viral pathogenic agent or a mammalian cell which is infected with a bacterial pathogenic agent.

Advantageously, the host cell according to the invention does not naturally express said first nucleic acid sequence (encoding an antibody). Preferably, a said cell is present in a form which enables it to be administered into the body of a (human or animal) mammal and, where appropriate, to be cultured beforehand, with said cell being genetically modified in vitro by introducing:

at least one said first nucleic acid sequence encoding all or part of an antibody, characterized in that, when this said antibody or this said antibody part is expressed, it is located on the surface of said host cell and in that said antibody or said antibody part is able to bind to a polypeptide which is present on the surface of a cytotoxic effector cell or of a T helper lymphocyte, with said polypeptide being involved in the process of activating such a cell, and

at least one said second nucleic acid sequence encoding all or part of a polypeptide which permits activation of the immune response and/or the chemoattraction of a cytotoxic effector cell and/or of a T helper lymphocyte. According to a preferred embodiment, said polypeptide is selected from chemokines and costimulation molecules, and

optionally, at least one third nucleic acid sequence encoding all or part of a polypeptide which possesses a cytotoxic activity.

More specifically, said host cell is derived either from the mammal to be treated or from a mammal which is different from the one to be treated. In this latter case, it should be noted that said host cell will have undergone a treatment which renders it compatible with the mammal to be treated. According to a preferred embodiment, “mammal” is understood as meaning a human mammal.

When it is administered to a patient, and more specifically administered by the intratumor route, such a biological material is able to induce or stimulate, in this patient, a cell-mediated immune response which is able to lead to the production of cytokines and the cytotoxic effect of the effector cells, which production and effect result not only in the elimination of the cells which have been administered but also in the elimination of the neighboring cells which are presenting the antigens, in particular tumor antigens, which are capable of being recognized by said activated cytotoxic effector cells.

The invention also relates to a method for preparing a cell according to the invention, characterized in that said first and second nucleic acid sequences, as described above, and, optionally, said third nucleic acid sequence, are introduced, using any appropriate means, into a mammalian cell, with said nucleic acid sequences being placed under the control of elements which ensure that they are expressed in said host cell, and in that the cells which have been genetically modified with said nucleic acid sequences are then selected from among these cells.

The invention furthermore relates to the use of a biological material or of a cell according to the invention for preparing a pharmaceutical composition which is intended for treating or preventing cancers or viral infections. More specifically, the invention concerns the co-utilization of, on the one hand, a nucleic acid sequence which encodes all or part of an antibody which is expressed on the surface of said target cell and which is able to bind to a polypeptide, which is present on the surface of a cytotoxic effector cell or of a T helper lymphocyte and which is involved in the process of activating such a cell, and of, on the other hand, a second nucleic acid sequence which encodes a polypeptide, which is of the chemokine or costimulation molecule type and which is involved in stimulating the immune response or in attracting to the expression site, and activating, cytotoxic effector cells or T helper lymphocytes, for preparing pharmaceutical compositions which are intended for treating a mammal by means of gene transfer. The invention also relates to the use of said first and second nucleic acid sequences and of a third nucleic acid sequence which encodes a polypeptide which possesses a cytotoxic activity. A preferred use relates (i) to an MVA vector or an MVA viral particle which comprises a nucleic acid sequence encoding the antibody KT3, which is expressed in a transmembrane manner, (ii) to an adenoviral vector or an adenoviral particle which comprises a nucleic acid sequence encoding the Act-2 variant of the chemokine MIP-1β and (iii) to an adenoviral vector or an adenoviral particle which comprises a nucleic acid sequence encoding IL-2 or IL-12.

For the purpose of implementing the therapeutic use which is mentioned in the present invention, it is possible to have available pharmaceutical compositions which comprise a biological material or a cell, as previously described, advantageously associated with a vehicle which is pharmaceutically acceptable for administering to humans or to an animal. The use of such vehicles is described in the literature. This pharmaceutically acceptable vehicle is preferably isotonic or hypotonic or exhibits weak hypertonicity and a relatively low ionic strength, such as, for example, a sucrose solution. Furthermore, said composition can contain solvents, aqueous or partially aqueous vehicles, such as pyrogen-free sterile water, and dispersion media, for example. The pH of these pharmaceutical compositions is appropriately adjusted and buffered, using conventional techniques.

According to a first possibility, the drug can be administered directly in vivo (for example into an accessible tumor or at its periphery, by the intravenous route or into the vascular system using an appropriate catheter). It is also possible to adopt the ex vivo approach, which consists in removing host cells from the mammal to be treated (bone marrow cells, peripheral blood lympocytes, etc.), transfecting or infecting them in vitro using the techniques of the art and then readministering them to said mammal.

The biological material according to the invention can be administered in vivo, in particular in injectable form, in particular by the intratumor route. It is also possible to envisage injection by the intratracheal, intranasal, epidermal, intravenous, intraarterial, intracardiac, intramuscular, intrapleural, intraperitoneal or intracerebral route using a syringe or any other equivalent means. According to another embodiment, it is possible to use systems which are adapted for treating the airways or the mucous membranes, such as inhalation, installation or nebulization, by the topical route, by oral administration or by any other means which is well-known to the skilled person and which can be applied to the present invention. Administration can take place in a single dose or in a dose which is repeated, once or more than once after a certain interval of time. The most appropriate routes of administration and doses vary in dependence on a variety of parameters such as the individual or the disease to be treated, or else the nucleic acid to be transferred or the host organ/tissue. By way of indication, the viral particle-based compositions can be formulated in the form of doses of between 10⁴ and 10¹⁴ iu (infectious units) or pfu (plaque-forming units), preferably from 10⁶ to 10¹² iu or pfu and, altogether preferably, from 10⁷ to 10¹¹ iu or pfu. Doses comprising from 0.01 to 100 mg of DNA, preferably from 0.05 to 10 mg of DNA and, altogether preferably, of from 0.5 to 5 mg of DNA, can be envisaged in the case of plasmid vector-based compositions.

A preferred use consists in treating or preventing cancers, tumors and diseases which result from unwanted cell proliferation. Applications which can be envisaged and which may be mentioned are cancers of the breast, of the uterus (in particular, those induced by papillomaviruses), of the prostate, of the lung, of the bladder, of the liver, of the colon, of the pancreas, of the stomach, of the esophagus, of the larynx, of the central nervous system and of the blood (lymphomas, leukemia) . The biological material can also be used within the context of cardiovascular diseases, for example for inhibiting or retarding the proliferation of the smooth muscle cells of the blood vessel wall (restenosis). Finally, application to AIDS, hepatites and cancers induced by viruses (retroviruses, papillomaviruses, etc.) can be envisaged in the case of infectious diseases. A composition according to the invention is more specifically intended for the preventive or curative treatment of diseases by gene therapy and is more specifically aimed at the aforementioned proliferative diseases and diseases of infectious origin.

According to an advantageous embodiment, the pharmaceutical composition of the invention can be used in combination with at least one compound which is naturally responsible for costimulating cytotoxic effector cells or T helper lymphocytes. According to this embodiment, it is possible to envisage administering said composition together with a polypeptide of the cytokine or chemokine type, for example IL-2 or IL-12.

The invention also extends to a method for treating and preventing diseases by means of gene transfer (gene therapy), characterized in that a biological material or a host cell according to the invention is administered to an organism or a host cell, in particular a mammalian organism or host cell, which is in need of such treatment. When the treatment method makes use of a biological material or of a host cell which comprises a third nucleic acid sequence, encoding a cytotoxic gene of the suicide gene type, the treatment method also comprises an additional step according to which quantities, which are acceptable from a pharmaceutical point of view, of the prodrug which acts in concert with the selected suicide gene are administered. In the case of a gene encoding the enzyme CDase and/or UPRTase, preference is given to using a cytosine analog such as 5-FC. By way of indication, it is possible to use a dose of from 50 to 500 mg/kg/day, with a dose of 200 mg/kg/day being preferred. Within the context of the present invention, standard practices are used for administering the prodrug, with this administration being before, concomitantly with or after that of the therapeutic agent according to the invention. The oral route is preferred. It is possible to administer a single dose of the prodrug or doses which are repeated for a time which is sufficiently long to enable the toxic metabolite to be produced within the organism or the host cell.

According to an advantageous embodiment, the therapeutic use or the treatment method can be combined with a second treatment of the patient. by means of surgery (in particular for ablating the tumor partially or totally), by means of radiotherapy or by means of chemotherapy. In this particular case, the treatment according to the invention is administered either before, concomitantly with or after said second treatment. Preference is given to this treatment being administered after said second treatment.

Furthermore, in order to improve the antineoplastic effect, the therapeutic use or the treatment method according to the invention can be combined with an additional treatment of the patient with compounds which are aimed at reducing the inflammatory response which is induced at the tumor site (for example a vasoactive compound such as serotonin) or a compound which inhibits the reactive forms of oxygen (for example histamine). In this particular case, the treatment according to the invention is administered either before, concomitantly with or after said additional treatment of the patient.

The contents of the prior art cited in the present application, including the published patent applications, the patents, the publications and the sequences identified by a database accession number, is hereby incorporated into the present application by reference.

The purpose of the examples which follow is to illustrate the various parts of the subject matter of the present invention and these examples are consequently in no way limiting.

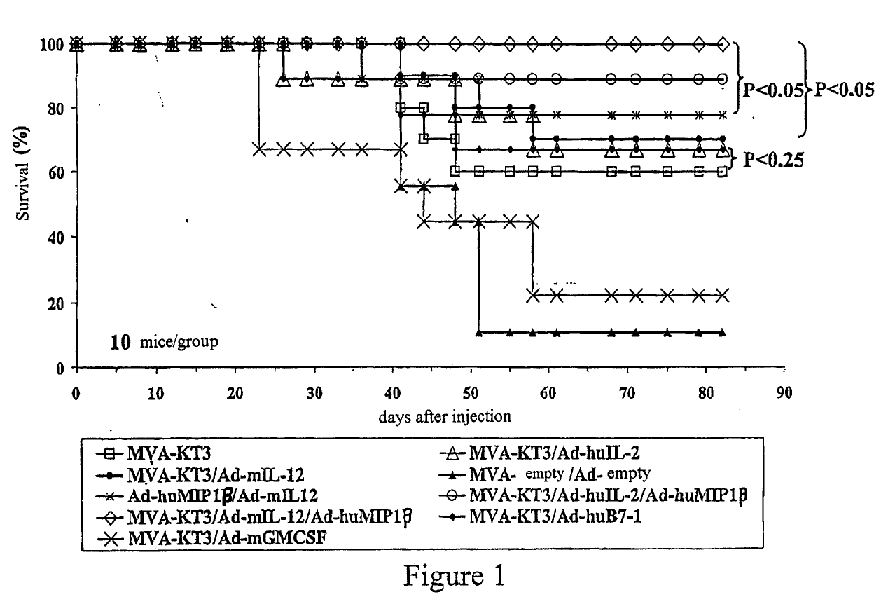

FIG. 1 is a diagram of the combined strategies which make use of the MVA-KT3 vector and the Ad-cytokine vectors and/or Ad-chemokine vectors in the RenCa model. The injection protocol consists of 2×10⁸ iu of adenovirus being injected on D0, D2 and D4 and 2×10⁷ pfu of MVA virions being injected on D2, D3 and D4. The statistical values are calculated between 2 curves which are linked by a bracket (Statistica 5.1, Mat&Met software). A value of p<0.05 is regarded as being statistically significant.

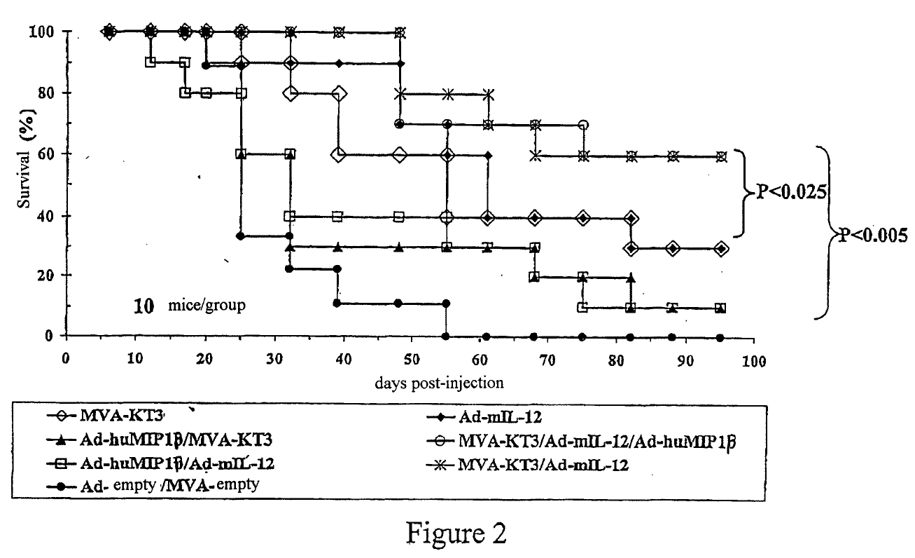

FIG. 2 is a diagram of the combined strategies making use of the MVA-KT3 vector and the Ad-cytokine vectors and/or Ad-chemokine vectors in the RenCa model. The injection protocol consists of 1×10⁸ iu of adenovirus being injected on D0, D1 and D2 and 1×10⁷ pfu of MVA virions being injected on D2, D3 and D4. The statistical values are calculated between the 2 curves which are linked by a bracket (Statistica 5.1, Mat&Met software). A value of p<0.05 is regarded as being statistically significant.

### EXAMPLES

The constructions which are described below are effected in accordance with the conventional techniques of genetic engineering and molecular cloning, as detailed in Maniatis et al., (1989, Laboratory Manual, Cold Spring Harbor, Laboratory Press, Cold Spring Harbor, N.Y.) or in accordance with the manufacturer's recommendations when a commercial kit is being used. The homologous recombination steps are preferably carried out in the E. coli strain BJ 5183 (Hanahan, 1983, J. Mol. Biol., 166, 557-580). The technique employed for repairing restriction sites consists in filling in the 5′ protruding ends using the large fragment of E. coli DNA polymerase I (Klenow fragment). In addition, the adenoviral genome fragments which are used in the different constructions described below are indicated precisely in accordance with their position in the nucleotide sequence of the Ad5 genome as disclosed in the Genbank database under reference M73260.

As far as cell biology is concerned, the cells are transfected or transduced and cultured using the standard techniques which are well known to the skilled person.

I. Tumor Models

Three tumor cell models were selected for the purpose of assessing the activity of the composition of the invention: P815 (H-2d mastocytoma, described in Dunn et al., 1957, J. Natl. Cancer Inst., 18, 587-590), B16FO (H-2b melanoma, described in Wu et al., 1996, Cancer Res., 56, 21-26) and RENCA (H-2d renal carcinoma, described in Murphy et al., 1973, J. Natl. Cancer Inst., 50, 1013-1025).

In order to establish the tumors in mice, the tumor cells P815, B16F10 or RENCA are trypsinized, washed 3 times in PBS and resuspended at 3×10⁶ cells/ml. 100 μl of this cell suspension are then injected into the right flank of immunocompetent B6D2 [(C57BL/6×DBA/2)F1] mice of 6-7 weeks of age. After the appearance of a tumor of palpable volume of between 5 and 25 mm³, each mouse is given three intratumor injections (100 μl) of a defined quantity of virus in 10 mM tris-HCl, pH 7.5, 1 mM MgCl₂. Each condition is assessed in groups of 10 mice. The tumor volume and the survival of the mice are determined 2 times each week. After a primary rejection, the mice are reinjected into the opposite flank using the same dose of tumor cells (3E+5 cells/mouse).

The efficacy of the composition of the invention which has been administered is checked by measuring the size of the tumors and by measuring the survival time of the treated mice together, where appropriate, with a verification of the immunological status of the animal using ELISPOT, a CTL test, etc. The animals can then be subjected to a contralateral challenge in which a lethal dose of tumor cells is administered to the pretreated animal.

### Example 1

### Construction and Functional Ability of the Vectors According to the Invention

1. Constructing a Recombinant MVA which Carries the First Nucleotide Sequence Encoding the Antibody Kt3, which is Expressed in Membrane Form and is Able to Bind the TCR/CD3 Complex which is Present on the Surface of the T Cells (See WO 00/24896).

The cloning of the sequences encoding the whole of the heavy and light chains of the antibodies KT3 and H57 is described in International application WO 00/24896. The chains which have thus been isolated are subcloned, by recombination, into a recombinant MVA virus which encompasses the nucleic acid sequence encoding the transmembrane region of the rabies virus (modified Ankara vaccinia; Antoine et al., 1998, Virology, 244, 365-396 and French patent application FR 97 09152) in order to obtain the virus MVATG14240, expressing the rat antibody KT3 (anti-rat CD3 epsilon), and MVATG14237, expressing the hamster antibody H57-597 (anti-hamster TCR alpha/beta). The expression cassettes are introduced into the MVA deletion II, as described in application WO 00/24896. Briefly, the light chain of the antibodies is placed under control of the early late p7.5 promoter (Goebel et al., 1990, Virol., 179, 247-266). The sequence encoding the heavy chain is placed under control of the early late pH5R promoter (Goebel et al., 1990, Virol., 179, 247-266). The C-terminal end of the heavy chain is fused to the transmembrane and intracytoplasmic domain of the rabies glycoprotein in order to enable the antibody to be anchored in the plasma membrane.

Production of the antibodies KT3 and H57 in the infected cells is verified by means of Western blotting and flow cytometry. The results which are observed show that rat and hamster type IgG immunoglobulin is indeed expressed on the surface of the infected cells (see example 2 in WO 00/24896).

The functional ability of the MVA viruses expressing the anti TCR/CD3 antibodies is tested by means of proliferation tests carried out on murine spenocytes. Expression of the KT3 or H57 antibody on the surface of murine cells induces strong proliferation of naive T cells (see example 2 in WO 00/24896).

### Example 2

### Construction and Functional Ability of the Recombinant Adenoviruses

1. Constructing the Recombinant Adenoviruses.

The human chemokine genes which were used were reconstituted by assembling oligonucleotides in accordance with their sequence as published in the “National Center for Biotechnology Information” database. This cloning strategy consists in assembling 8 oligonucleotides, in each case containing approximately 80 bases, in two steps. The whole assembly covers the totality of the nucleotide sequence of the cDNA for the genes Act-2 variant of MIP-1β (accession: J04130, described in application WO 00/74629, termed MIP-1β below), IP-10 (accession no.: X02530) and DC-CK1 (accession no.: AB000221). The cloning of the genes encoding human IL-18 (accession no.: 4504652) and human B7-H1 (accession no.: AF177937) was effected by means of RT-PCR using human macrophage RNA. IL-18 does not possess any natural signal sequence and is only produced in the mature protein state after having been cleaved with caspase-1. The sequence encoding the mature protein (nucleotides 108-582) was therefore cloned in phase with the previously described signal peptide BM40 (Yamaguchi et al., 1999, EMBO J., 18, 4414-1423). The chemokine BRAK was cloned on the basis of the sequence described by Frederick et al. (2000, Am. J. Pathol., 156, 1937-50) and Sleeman et al. (2000, Int. Immunol., 12, 677-689). These genes were cloned into an adenoviral transfer vector.

The transfer vectors expressing the genes for human IL-2, two subunits of murine IL-12 (p40 and p35), separated by an internal ribosome entry site, mGM-CSF (human leader sequence in phase with the murine sequence) and murine B7-1 were constructed by inserting the sequence, cloned on the basis of the published sequence, into the transfer vector.

The transfer vector consists of an expression cassette which contains the promoter/enhancer sequences of human cytomegalovirus (CMV), a chimeric human β-globin/IgG intron and an SV40 virus polyadenylation sequence. The sequences flanking the E1 region (5′ fragment, nucleotides 1 to 458, and 3′ fragment, nucleotides 3328 to 5788) of human adenovirus type 5 were placed at either end of this cassette. These sequences make it possible to generate an “infectious” plasmid by means of homologous recombination, in E. coli, with the complete ΔE1/ΔE3 adenoviral genome containing the candidate gene (Chartier et al., 1996, J. Virol., 70, 4805-4810). The recombinant adenoviruses are then produced by transfecting a PacI digest of the “infectious” plasmid into a complementation cell line (Graham et al., 1977, J. Gen. Virol., 36, 59-74). A recombinant adenovirus which does not contain any transgene in the expression cassette (Ad-empty) serves as the control adenovirus in all the experiments. The virions were propagated, purified and titrated using standard techniques (Lusky et al., 1998, J. Virol., 72, 2022-32). The purified viruses are stored at −80° C. in 1M sucrose, 10 mM tris-HCl, pH 8.5, 1 mM MgCl₂, 150 mM NaCl, 0.005% Tween 80.

2. Cell Lines and Primary Cells.

The murine tumor cell lines P815, B16F0 and B16F10 were obtained from the ATCC (American Type Culture Collection; Rockville, Md., USA). The P815 cells (ATCC No.: TIB-64) are derived from a DBA/2 (H2-K^{d}) mouse mastocytoma. The highly metastatic B16F10 cells (ATCC, CRL-6475) are derived from a C57BL/6J (H2-K^{b}) mouse melanoma. The RenCa cells are derived from a murine (BalB/cCr (H2-K^{d})) renal carcinoma. The tumor cell line A549 (ATCC, CCL-185) is derived from a human pulmonary carcinoma. Human monocytes are obtained by leukapheresis and elutriation. The cells were cultured, at 37° C. and 5% CO₂, in DMEM medium (Gibco-BRL) which was supplemented with 10% fetal calf serum (FCS). The human dermal microvascular cells (HDMECs) were cultured as recommended by the supplier (PromoCell, Heidelberg, Germany).

3. Analyzing Protein Expression in Vitro.

The presence of the proteins huIL-18 and MIP-1β was analyzed on culture supernatants obtained from A549 cells which have been infected for 48 h at a multiplicity of infection (MOI) of 5. The supernatants were quantified by immunoassay (Quantikine; R&D Systems, Minneapolis, USA). The in vitro expression of the proteins huIP-10, huMIP-1β and huIL-18 was analyzed on culture supernatants from HDMEC cells which had been infected for 48 h at an MOI of 50. This is because the supernatants from HDMEC cells are much less rich in contaminating proteins than are those from other cell lines. The recombinant compounds were quantified on a 12% polyacrylamide gel (SDS-PAGE) stained with Coomassie Blue (Neuhoff et al., 1988, Electrophoresis, 9, 255-62).

4. Assessing the in Vitro Functional Ability of the Recombinant Adenoviruses.

The chemotaxis test derives from the method published by Senger et al. (1983, Science, 219, 983-5), using modified Boyden migration chambers. The migration chamber membranes of 6.5 mm in diameter (TRANSWELL Corning Costar Corporation, Badhoevedorp, Netherlands), containing 5 μm pores, are reversed and covered with collagen (SIGMA-Aldrich, Saint-Quentin Fallavier, France) in order to prevent passage of the cells (100 μg/ml). After 60 minutes of polymerization at ambient temperature, the chambers are rinsed in PBS. The membranes are then saturated with bovine serum albumin (BSA; 100 mg/ml; Sigma Alldrich), incubated for 60 minutes at ambient temperature and then rinsed in PBS. The supernatants from the infected cells are supplemented with 10 mg of BSA/ml, then deposited in the wells at a rate of 300 μl/well and covered with a cupule. 300 μl of a suspension of 3×10⁵ human monocytes/ml are added to the upper part of the cupule. After 4 hours of incubation, at 37° C. and 5% CO₂, the cupules are removed from the wells, the supernatant is aspirated and the membrane is cleansed twice with PBS. It is then stained with a solution of 0.2% crystal violet, 2% ethanol for 5 minutes, and rinsed with PBS, and the number of cells present is determined using an optical microscope.

5. Costimulating by Means of the Membrane Expression of B7-H1.

The proliferation of naive splenocytes which is induced by the costimulating compound B7H-1 was assessed using the technique described in Dong et al. (1999, Nat. Med, 5, 1365-1369). Briefly, the RENCA cells are infected, at an MOI of 50, with Ad.huB7H-1 and then treated for 1 hour with 50 μg of mitomycin C/ml (SIGMA-Aldrich, Saint-Quentin, Fallavier, France). In parallel naive splenocytes are prepared from a DBA/2 mouse spleen (Paul et al., 1999, Cancer Immunol. Immunother., 48, 22-28). After washing, the tumor cells and the splenocytes are cocultured. The positive and negative controls consist of splenocytes which have been activated with 10 μg of ConA/ml and which have not been stimulated, respectively (R&D Systems, Minneapolis, USA) . After 96 hours at 37° C. and 5% CO₂, 1 μCi of tritiated [³H] thymidine is added/well. The quantity of thymidine which has been incorporated is measured after 8 hours by precipitating the cell DNA onto glazed filter paper (PHD harvester, Cambridge Technology, Plainfield, USA). The radioactivity which is emitted is measured using a β counter (Beckman Instruments Inc., Palo Alto, USA).

6. Assessing the Combinatorial Strategies in Vivo.

In order to establish tumors in mice, the tumor cells P815, B16F10 or RENCA are trypsinized, washed 3 times in PBS and resuspended at a concentration of 3×10⁶ cells/ml. 100 μl of this cell suspension are then injected into the right flank of immunocompetent B6D2 [(C57GBL/6×DBA/2)F1] mice of 6-7 weeks of age. After the appearance of a tumor of palpable volume of between 5 and 25 mm³, each mouse is given three intratumor injections (100 μl) of a defined quantity of virus in 10 mM tris-HCl, pH 7.5, 1 mM MgCl₂. Each condition is assessed in groups of 10 mice. Tumor volume and survival of the mice are determined twice weekly. For ethical reasons, the animals are sacrificed when the volume of the tumor becomes greater than or equal to 3000 mm³. After a primary rejection, the mice are reinjected with the tumor cells (3×10⁵ cells/mouse) on the opposite flank. Statistical studies carried out on the figures obtained make it possible to plot a survival curve of the Kaplan-Meier type. Statistical significance is calculated using Fisher's exact test (Statistica 5.1 software-Statsoft Inc., Tulsa, USA).

Results.

7. Functional Ability of the Adenoviral Vectors in Vitro.

7.1 Analyzing the Protein Expression in Vitro

Infection of these cells with adenoviruses encoding the cytokines huIL-2, mIL-12, huIL-18 and mGM-CSF enables secretion levels of between 20 ng and 7 μg/ml/10⁶ cells/48 h to be obtained. Polyacrylamide gel analysis of supernatants from HDMEC cells which have been harvested 48 h after infection with Ad-huMIP1β, Ad-huIP1β and Ad-huIL-18 results in the detection of bands of the expected size (MIP-1β (6.7 kDa), huIP-10 (8.7 kDa) and huIL-18 (18.2 kDa)).

7.2 Costimulating by Means of the Membrane Expression of B7-H1

The proliferation of syngenic naive splenocytes which was induced by the membrane molecule B7-H1 was assessed by measuring the incorporation of tritiated thymidine. To this end, RENCA cells were infected with Ad-empty or Ad-B7-H1 for 48 h and then treated with mitomycin C in order to stop their proliferation. The infected cells were then brought into contact with naive splenocytes (of the same haplotype as the RenCa cells) in order to assess whether the membrane expression of B7-H1 on the tumor cell surface induced proliferation of the lymphocytes. The results which were observed show that, while naive splenocytes proliferate (stimulation factor: 12×) in the presence of RenCa/Ad-B7-H1 cells, they do not proliferate in the presence of RenCa/Ad-empty cells, indicating that the Ad-B7-H1 vector functions in vitro.

7.3 Chemotactic Activity in Vitro

The functional ability of the chemokines/cytokines which were expressed by an adenoviral vector was assessed on human monocytes. The results which were observed show that the supernatants from A549 cells which have been infected with the vectors Ad-huMIP-1β and Ad-huDC-CK1 cause, respectively, 2.5 and 5 times more monocytes to migrate than does the control infection supernatant (Ad-empty). The infection supernatants obtained with the vectors Ad-muIL-12, Ad-huMIP-1α and Ad-huIP10 are ineffective in this test. Surprisingly, the supernatant obtained using Ad-huIL-18 makes it possible to chemoattract 13 times more monocytes than are chemoattracted by the control infection supernatant. This chemotactic activity of IL-18 has never been reported.

### Example 3

### In Vivo Antineoplastic Activity

With the aim of improving the antineoplastic efficacy of the vectors MVA-KT3 and MVA-H57 (see application WO 00/24896), these antibodies were coexpressed with cytokines, chemokines or costimulation molecules. The antineoplastic activity was determined in three mouse models which were implanted, respectively, with the tumors P815, B16F10 and RenCa.

Different immunotherapy protocols were assessed for the purpose of reducing to the maximum the information which was induced by injecting the two viruses. This is because coinjecting 2.10⁷ pfu of MVA-empty and 4.10⁸ iu of Ad-empty on D0, D1 and D2 eliminates the tumor in from 40 to 50% of the treated mice. In order to assess the antineoplastic efficacy of the combinations of molecules expressed, an immunotherapy protocol was established in order to determine the experimental conditions under which coinjecting the empty vectors did not induce tumor rejection in vivo or only induced such rejection to a slight extent. The protocol which was established consisted in injecting an adenoviral vector dose (from 1.10⁸ to 4.10⁸ iu depending on the tumor models) on D0, D1 and D2 and an MVA vector dose (1.10⁷ or 2.10⁷ pfu depending on the tumor models) on D2, D3 and D4. This protocol considerably reduces the antineoplastic activity which is induced by coinjecting the control viruses in the three tumor models. For example, in the RenCa model, the simple change consisting in firstly injecting the adenovirus reduces the percentage of mice which have rejected their tumor from 20 to 0 (comparison of FIGS. 1 and 2). The results obtained in these combined antineoplastic strategies are always compared with those obtained in the groups treated with the viruses on their own, in the case of the double combinations, and with those obtained in the groups treated with two out of the three viruses, in the case of the triple combinations.

1. Antineoplastic Effects of the Double Combinations.

Certain cytokines (IL-2, IL-12 and IL-18) have been reported to be compounds which activate effector cells of the NK or LAK type which are not MHC-restricted. The antineoplastic efficacy of these compounds could add to that of the vectors MVA-KT3 and MVA-H57. In the P815 model, coinjection of Ad-huIL-2 and MVA-H57 increases the survival of the animals from 30 days to 45 days as compared with the groups treated with MVA-H57 or Ad-huIL-2 on their own and statistically improves the survival of the animals (from 15 to 35-40% rejection, depending on the experiments). In the B16F10 model, the MVA-KT3+Ad-huIL-2 combination appreciably increases (from 10 to 20%) the number of mice having rejected their tumor as compared with the control groups. Finally, strong synergy between the anti-TCR/CD3 antibodies and the cytokines is observed in the RenCa model (FIGS. 1 and 2). In this model, the combination of MVA-KT3 and Ad-mIL-12 increases the number of animals which are cured as compared with the groups which are treated with only one out of the two vectors. Thus, as FIG. 1 shows, the survival rate of the treated animals changes from 60%, after administration of MVA-KT3 on its own, to 70%, after administration of MVA-KT3 and Ad-IL-12. According to the injection protocol which is used for the experiment shown in FIG. 2, the survival rate of the treated animals changes from 30%, after administration of Ad-IL-12 on its own, and 40%, after administration of MVA-KT3 on its own, to 60% after administration of MVA-KT3 and Ad-IL-12. This synergistic effect (p<0.005) can be reproduced with other injection protocols. Under the experimental conditions which were employed, it was not possible to demonstrate any synergy in the case of the other cytokines (GM-CSF and IL-18) which were tested.

The coinjection of adenoviral vectors expressing the costimulation compounds B7-1 and B7-H1 appreciably improves the antineoplastic efficacy of the MVA-KT3 vector in the RenCa and B16F10 models. Thus, the survival rate of the animals changes from 60%, after administration of MVA-KT3, to 65% after administration of MVA-KT3 and Ad-huB7-1 (FIG. 1).

The results obtained show that, in different murine tumor models, coinjecting an adenoviral vector encoding. IL-12 increases the antineoplastic effect which is induced by the in vivo membrane expression of the vector MVA-KT3. This synergy can be explained by several nonexclusive mechanisms. The apparent antineoplastic efficacy of IL-12 can be mediated by macrophages, NK cells (CD3⁻) and/or NKT cells (NK1.1⁺, CD3⁺, TCRαβ⁺, CD4⁺, CD8⁻, CD28⁺, Vα14). It is conceivable that, in the RenCa model, which is very permissive to an adenoviral infection and which probably secretes substantial quantities of cytokines, the synergistic antineoplastic effect of IL-12 and KT3 is a reflection of the activation of the NK (CD3⁻) cells by IL-12 and of the CD3⁺ cells (T and NKT) by the KT3 antibody. In the B16F10 model, which is less permissive to adenoviral infection (low quantities of cytokines), the antineoplastic effect of IL-12 could be mediated both by activation of the NK cells and activation of a larger number of NKT cells, due to the diffusion of the cytokine. Overexpression of MHCI and CD1d on the surface of these different tumor models could also exert an influence on the importance of the NK and NKT cells in the IL-12-mediated process of tumor rejection. Finally, the antiangiogenic activity of IL-12, mediated by induction of the chemokine IP-10, could reduce the growth of the tumor by inhibiting its neovascularization. This would enable the different immune effectors induced by IL-12 and KT3 [lacuna] on a tumor whose growth is slowed down.

2. Antineoplastic Effects of the Triple Combinations.

The antineoplastic effect of triple combinations was assessed by adding an adenoviral vector expressing chemokines to these double combinations of interest: the adenoviral vectors were administered on DO, Dl and D2 (from 1 to 2×10⁸ iu/vector/injection) and the vector MVA-KT3 was administered on D2, D3 and D4 (from 1 to 2×10⁷/injection). The injection of the Ad-huMIP1α+Ad-huIL-2+MVA-KT3 viruses slightly increases (from 15 to 25%) the survival of the animals in the P815 model. In the RenCa model, the triple combinations MVA-KT3/Ad-muIL-12/Ad-huMIP1β and MVA-KT3/Ad-huIL-2/Ad-huMIP1β respectively induce 100 and 90% survival in the treated mice (FIG. 1). In these experiments, a vector Ad-huMIP1β dose [lacuna] reduced by half as compared with that used when the Ad-chemokine and MVA-KT3 double combinations were employed.

In the RenCa model, the rate of survival of the animals was also increased after administering the triple combination MVA-KT3/Ad-huBRAK/Ad-huIL-2, reaching 75%, whereas the survival rates observed after administering Ad-huBRAK and Ad-huIL-2, Ad-huIL-2 on its own and MVA-KT3 on its own are, respectively, 63%, 50% and 20%.

Thus, the use of adenoviral vectors encoding a chemokine (MIP-1β or BRAK) and a cytokine (IL-12 or IL-2) in combination with the vector MVA- encoding the antibody KT3, expressed in transmembrane form, results in 75 to 100% of the animals being cured in the RenCa model (FIG. 1 and above). The efficacy of the triple combinations MVA-KT3+Ad-huMIP-1β+Ad-mIL-12 and MVA-KT3+Ad-huMIP1β+Ad-huIL-2 could be explained by the induction of a multifaceted mechanism which we have termed AAD (attraction, activation and death). In this model, injection of the MVA-KT3 vector induces activation of the CD3⁺ (T and NKT) cells and infiltration of dendritic cells at the tumor site. The injection of the adenoviral vectors Ad-huMIP1β and Ad-mIL-12 attracts and activates NK and NKT cells and macrophages in situ. Other combinations are also conceivable.

1. A biological material which comprises at least one first and at least one second nucleic acid sequence, said nucleic acid sequences being placed under the control of elements which ensure their expression in a host cell, wherein:
said first nucleic acid sequence encodes all or part of an antibody, characterized in that, when this said antibody or this said antibody part is expressed, it is located on the surface of said host cell, and in that said antibody or said antibody part is able to bind to a polypeptide which is present on the surface of a cytotoxic effector cell or of a T helper lymphocyte, said polypeptide being involved in the process of activating such a cell,
said second nucleic acid sequence encodes all or part of a polypeptide which is involved in the stimulation of the immune response and/or in the attraction to the expression site, and in the activation of the cytotoxic effector cells and/or T helper lymphocytes.

2. The biological material of claim 1, characterized in that it is in the form of naked DNA or RNA.

3. The biological material of claim 1, characterized in that said first nucleic acid sequence and/or said second nucleic acid sequence is comprised in a vector which enables it (them) to be transferred.

4. The biological material of claim 3, characterized in that said first and second nucleic acid sequences are comprised in independent vectors which enable said nucleic acid sequences to be transferred into said host cell.

5. The biological material of claim 3 or 4, characterized in that said vector or vectors is/are (a) viral vector(s)

6. The biological material of claim 5, characterized in that said viral vectors are adenoviral vectors, retroviral vectors or poxviral vectors, in particular derived from the vaccinia virus or from the modified Ankara virus (MAV) strain of the vaccinia virus.

7. The biological material of one of claims 4 to 6, characterized in that said first nucleic acid sequence is comprised in a poxviral vector which is derived from the modified Ankara virus (MAV) strain of the vaccinia virus and in that said second nucleic acid sequence is comprised in an adenoviral vector.

8. The biological material of claim 3 or 4, characterized in that said vectors are complexed or conjugated with at least one carrier substance or molecule which is selected from the group consisting of a cationic amphiphilic substance, in particular a cationic lipid, a cationic or neutral polymer, a protic polar compound, selected, in particular, from propylene glycol, polyethylene glycol, glycerol, ethanol or 1-methyl-L-2-pyrrolidone or their derivatives, and an aprotic polar compound selected, in particular, from dimethyl sulfoxide (DMSO), diethyl sulfoxide, di-n-propyl sulfoxide, dimethylsulfone, sulfolane, dimethylformamide, dimethylacetamide, tetramethylurea or acetonitrile or their derivatives.

9. The biological material of one of claims 1 to 8, characterized in that said first nucleic acid sequence contains a gene encoding the heavy chain of an antibody which is able to bind to a polypeptide which is present on the surface of a cytotoxic effector cell or of a T helper lymphocyte, and which is involved in the process of activating such a cell, with the sequence being fused to a peptide which is able to confer to said antibody a transmembrane localization.

10. The biological material of claim 9, characterized in that said first nucleic acid sequence additionally contains a gene encoding the light chain of an antibody which is able to bind to a polypeptide which is present on the surface of a cytotoxic effector cell or of a T helper lymphocyte and which is involved in the process of activating such a cell.

11. The biological material of claim 9 or 10, characterized in that said peptide which is able to confer a transmembrane localization is isolated from a glycoprotein, from a lipoprotein or from a membrane receptor.

12. The biological material of claim 11, characterized in that said glycoprotein is selected from the group consisting of the rabies virus glycoprotein, the measles virus F glycoprotein, the HIV virus gp160 and the CD4.

13. The biological material of one of claims 1 to 12, characterized in that said polypeptide which is present on the surface of a cytotoxic effector cell or of a T helper lymphocyte and which is involved in the process of activating such a cell is a receptor.

14. The biological material of claim 13, characterized in that said cytotoxic effector cell is selected from the group consisting of macrophages, cytotoxic T lymphocytes (CTLs) and killer cells (NKs) or their derived cells.

15. The biological material of claim 13 or 14, characterized in that said receptor is selected from the group consisting of all or part of the TCR complex, more specifically TCR-α, TCR-β or CD3, CD8, CD4, CD28, LFA-1, 4-1BB, CD47, CD2, CD9, CD45, CD30, CD40, the cytokines receptors such as IL-7, IL-4, IL-2, IL-12, IL-15, IL-18, IL-21 or GM-CSF, Vα14NKT, NKAR, NKp44 and the Fc receptor.

16. The biological material of one of claims 1 to 15, characterized in that said second nucleic acid sequence encodes all or part of a chemokine selected from RANTES, MIG, IL-8, MCP-1, BRAK, MIP-1α, MIP-1β and MIP-2.

17. The biological material of one of claims 1 to 15, characterized in that said second nucleic acid sequence encodes all or part of a costimulation molecule selected from B7-1 and B7-H1.

18. The biological material of one of claims 1 to 17, characterized in that said second nucleic acid sequence encodes all or part of a polypeptide secreted by said host cell.

19. The biological material of one of claims 1 to 18, characterized in that it additionally comprises a third nucleic acid sequence encoding all or part of a polypeptide having cytotoxic activity.

20. The biological material of claim 19, characterized in that said polypeptide having cytotoxic activity is IL-2 or IL-12.

21. The biological material of claim 20, characterized in that:
said first nucleic acid sequence encodes the transmembrane peptide of the rabies virus glycoprotein, the heavy chain and light chain of the antibody KT3,
said second nucleic acid sequence encodes the chemokine MIP-1β or BRAK, and
said second third nucleic acid sequence encodes IL-2 or IL-12.

22. A host cell, comprising a biological material of one of claims 1 to 21.

23. The host cell of claim 22, characterized in that said host cell is a mammalian tumor cell, a mammalian cell which is infected with a viral pathogenic agent or a mammalian cell which is infected with a bacterial pathogenic agent.

24. The host cell of claim 22 or 23, which does not naturally express said first nucleic acid sequence (encoding an antibody) in a form which enables it to be administered into the body of a mammal and, where appropriate, to be cultured beforehand, and said cell being genetically modified in vitro by introducing:
at least one said first nucleic acid sequence encoding all or part of an antibody, characterized in that, when this said antibody or this said antibody part is expressed, it is located on the surface of said host cell and in that said antibody or said antibody part is able to bind to a polypeptide which is present on the surface of a cytotoxic effector cell or of a T helper lymphocyte, said polypeptide being involved in the process of activating such a cell, and
at least one said second nucleic acid sequence encoding all or part of a polypeptide which allows the activation of the immune response and/or the chemoattraction of a cytotoxic effector cell and/or a T helper lymphocyte,
and, optionally, at least one third nucleic acid sequence encoding all or part of a polypeptide having cytotoxic activity.

25. The host cell of one of claims 22 to 24, characterized in that it is derived from the mammal to be treated.

26. The host cell of one of claims 22 to 24, characterized in that it is derived from a mammal which is different from that to be treated and has undergone a treatment rendering it compatible.

27. A method for preparing cells of claim 24, characterized in that said first and second nucleic acid sequences and, optionally, said third nucleic acid sequence are introduced into a mammalian cell using any appropriate means, said nucleic acid sequences being placed under the control of the elements which ensure their expression, and in that those cells which have been genetically modified with said nucleic acid sequences are then selected from these cells.

28. The use of a biological material of one of claims 1 to 21, or of a cell of one of claims 22 to 26, for preparing a pharmaceutical composition which is intended for treating or preventing cancers or viral infections.

29. A pharmaceutical composition which comprises a biological material of one of claims 1 to 21 or a cell of one of claims 22 to 26, advantageously in combination with a pharmaceutically acceptable vehicle.

30. A pharmaceutical composition of claim 29, which additionally comprises at least one compound which is naturally responsible for costimulating the cytotoxic effector cells or T helper lymphocytes.
